(19) United States
(12) Patent Application Publication
(10) Pub. No.: US 2023/0285710 A1
(43) Pub. Date: Sep. 14, 2023

(54) APPARATUS FOR SUPPLYING NO TO TWO GAS OUTLETS

(71) Applicant: INOSYSTEMS, Antony (FR)

(72) Inventor: Frederic MARCHAL, Antony (FR)

(21) Appl. No.: 18/119,204

(22) Filed: Mar. 8, 2023

(30) Foreign Application Priority Data
Mar. 9, 2022 (FR) 2202053

(51) Int. Cl. A61M 16/12; A61M 16/20

(52) U.S. Cl. CPC A61M 16/122; A61M 16/202; A61M 2205/502; A61M 2016/0033

(57) ABSTRACT

Disclosed is an NO supply device including a main gas circuit to convey an NO/N₂ mixture to a main output orifice which is designed to supply the inspiratory branch of a medical ventilator; and a backup circuit including a backup NO circuit including a backup NO line which is supplied with NO/N₂ mixture, and a backup O₂ circuit includes a main O₂ line which is connected fluidically to the backup NO line in order to form a common line in fluid communication with a secondary output which supplies an NO/N₂/O₂ mixture designed to supply a manual breathing bag.

### CROSS-REFERENCE TO RELATED APPLICATION

This application claims priority to FR 2202053 filed Mar. 9, 2022, the entire contents of which are hereby incorporated by reference.

### BACKGROUND OF THE INVENTION

### Field of the Invention

The present invention relates to a device or apparatus for supplying gaseous nitric oxide (NO) equipped with a backup system in order to ensure a supply of NO, in normal mode, in backup mode in the event of a fault, or in manual ventilation mode, in particular in the case of transition to manual ventilation by means of a breathing bag, or manual insufflation (i.e. BAVU), and an installation for administering NO to a patient comprising a device or apparatus of this type for supplying NO.

### Description of the Related Art

Inhaled nitric oxide, or NOi, is a gaseous medicament commonly used to treat patients suffering from acute pulmonary arterial hypertension, particularly pulmonary vasoconstrictions in adults or children, including the newborn (PPHN), as described for example in EP-A-560928 or EP-A-1516639.

An installation for implementation of treatment by NOi, commonly known as an installation for administering NO, conventionally comprises one or more cylinders of NO/N₂ mixture supplying a device for supplying NO, which supplies the NO/N₂ mixture with a controlled flow rate, a respiratory assistance apparatus, also known as a medical ventilator, for supplying a respiratory gas containing at least 21% by volume of oxygen, such as an O₂/N₂ mixture or air, to which the NO is added (i.e. NO/N₂), circuit elements, for example one or more flexible ducts, to convey the gaseous flows between these different items of equipment and to the patient, and a respiratory interface, such as an endotracheal tube, to supply the gaseous mixture containing the NO to the patient. It is also possible to provide a gas humidifier in order to humidify the gaseous mixture before it is administered to the patient. An installation of this type is schematised in FIG. 1.

Habitually, the NO/N₂ mixture which is supplied by the device for supplying NO is injected into the respiratory flow containing at least 21% by volume of oxygen (i.e. air or O₂/N₂ mixture) which is obtained from the medical ventilator before being administered by inhalation to the patient in the form of a final respiratory mixture (i.e. NO/N₂/O₂ or NO/N₂/air mixture) generally containing a few tens of ppmv of NO (ppm in volume) and at least 21% by volume of oxygen O₂, for example approximately 1 to 80 ppmv of NO, the remainder being substantially nitrogen (N₂).

An installation of this type for administering NO is used in a hospital environment in order to administer the treatment by NOi, and thus care for patients needing to inhale NO to treat their pulmonary arterial hypertension. Examples of such NO administration installations are given in documents WO-A-2012/094008, US-A-2015/320951, US-A-2015/273175, JP-A-H11192303, WO-A-O_{2/40914} and US-A-2003/116159.

In the event of a fault in, or failure of, the device for supplying NO, or of the medical ventilator, and/or if it is necessary to ventilate the patient with a manual breathing bag, at the request of the medical staff, or further to a slight fault of the device, the NO supply device must be able to continue to supply the NO/N₂ mixture so that the treatment of the patient is not interrupted suddenly, for obvious reasons of safety, since a sudden interruption of the treatment by NOi could be fatal for fragile patients, in particular newborns suffering from PPHN.

In order to ensure that NO is supplied even in the case of a fault, of ventilation by a manual breathing bag or other means, it is known to equip the NO supply device with a secondary system or circuit, known as the backup circuit, which is entirely pneumatic and is designed to supply a flow rate of O₂ which is adjustable between 0 and 20 L/min and a fixed flow rate of NO, for example approximately 230 mL/min of NO/N₂ mixture, which are mixed with one another in the NO supply device before being injected into a manual breathing bag. The oxygen is typically provided by a source of oxygen, such as a pressurised oxygen cylinder, connected to the NO supply device. Thus, reference can be made to EP-A-3233171 which teaches the use of a backup circuit of this type.

The transition to “backup mode” takes place after the user has activated a selection means present on the NO supply device, for example a button for selection of the “backup mode”, the activation of which will direct the flows of gas into a secondary circuit, i.e. a circuit for backup and/or manual ventilation of the NO supply device, such as the one illustrated in FIG. 2.

However, a solution of this type which is purely pneumatic is not ideal, since the concentration of NO of the gaseous mixture obtained (i.e. NO/N₂+O₂) varies according to the flow rate of oxygen regulated by the user, since the flow rate of NO is fixed. However, having a concentration of NO which varies according to the flow rate of O₂ selected is not desirable from the therapeutic point of view, since the patient must be able to be provided with a given dose, i.e. a fixed quantity of NO corresponding to a concentration which is efficient for treating the pathology of the patient, or a reduced dosage if withdrawal of the patient is necessary, in particular in manual ventilation mode via a manual insufflator, i.e. a manual breathing bag or BAVU.

In view of this, a problem consists of proposing an improved NO supply device, which can supply a NO/N₂/O₂ gaseous mixture to a patient, both in normal operating mode but also in the case of a fault or failure, or also during transition to manual ventilation by BAVU (e.g. at the request of the medical staff or further to a slight fault), preferably complying with the required dose, i.e. a NO/N₂/O₂ gaseous mixture, the composition of which is not dependent only on the flow rate of oxygen.

### SUMMARY OF THE INVENTION

A solution according to the invention concerns a device or apparatus for supplying NO (i.e. nitric oxide), comprising:
a main gas circuit comprising at least one main NO line connecting at least one NO input port fluidically to a main output orifice, in order to convey a NO/N₂ mixture of the said at least one NO input port to the said main output port; and
a backup circuit comprising a backup NO circuit and a backup O₂ circuit, wherein:
the backup NO circuit comprises a backup NO line which is supplied with NO/N₂ mixture obtained from the said at least one NO input port; and
the backup O₂ circuit comprises a main O₂ line which is in fluid communication with an O₂ input port, the main O₂ line being connected fluidically to the backup NO line in order to form a common line which is in fluid communication with a secondary output, also known as the backup output, supplying an NO/N₂/O₂ mixture, in particular such as to be able to supply to a manual breathing bag (BAVU) or the like.

In addition, the main NO line comprises means for controlling the flow rate of NO/N₂ which are controlled by control means, and the main NO line is connected fluidically to the backup NO line, downstream from the means for controlling the flow rate of NO/N₂, by means of a first solenoid valve with a plurality of ways.

According to the embodiment concerned, the device or apparatus for supplying NO according to the invention can comprise one or more of the following characteristics:
the control means (i.e. the control device) are supplied with electrical energy (i.e. electric current) by means for supplying electrical energy;
the main NO line is connected fluidically to the backup NO line by means of a first solenoid valve with at least 3 ways, preferably with 3 ways or 5 ways, advantageously with 3 ways;
the first solenoid valve with a plurality of ways comprises at least one input way which is connected fluidically to the main NO line downstream for the means for controlling the flow rate, and a first output way which is connected to the main NO line, in particular to the downstream portion of the main NO line comprising the main output orifice, and a second output way which is connected to the backup NO line upstream from the common line comprising the secondary orifice;
the first solenoid valve is controlled by the control means such as to direct the flow of NO/N₂ mixture obtained from the NO/N₂ flow rate control means 202 i.e. the flow rate control device:
   either into a downstream portion of the backup NO line which is in fluid communication with the common line in fluid communication with the secondary output;
   or into a downstream portion of the main NO line which is in fluid communication with the main output orifice;
the backup NO line comprises a pneumatic valve which makes it possible to control the circulation of the NO/N₂ mixture in the backup NO line;
the backup NO line comprises a secondary solenoid valve which is controlled by the control means;
the second solenoid valve of the backup NO circuit is normally open;
the main O₂ line comprises a first control valve controlled by means for actuation which can be actuated by a user in order to control the circulation of the flow of oxygen in the said main O₂ line;
the means for actuation (i.e. the actuating device) is configured to supply an activation signal to the control means in response to actuation of the said means for actuation by the user;
the main O₂ line comprises a second pneumatic control valve which is arranged downstream from the first control valve;
the control means are configured to control the NO/N₂ flow rate control means such as to regulate the flow rate of the NO/N₂ mixture in the main gas circuit;
the control means are configured to control the first solenoid valve in response to the receipt of the actuating signal supplied after actuation of the means for actuation by the user, for example in the case of a slight fault which does not give rise to total stoppage of electrical energy supply and/or stoppage of operation of the control means, or in particular when the user wishes to put into effect manual ventilation by means of BAVU;
the control means are configured to control the second solenoid valve of the backup NO circuit in order to interrupt any circulation of NO/N₂ mixture through the said second solenoid valve;
the first solenoid valve with a plurality of ways is arranged on the main NO line downstream from the NO/N₂ flow rate control means, and is in fluid communication with the backup NO line downstream from the pneumatic valve and the second solenoid valve;
the main O₂ line also comprises means for measurement of the flow of oxygen which are arranged downstream from the first control valve, and are configured to provide at least one measurement of flow rate of oxygen to the control means;
the main O₂ line also comprises means for regulation of the flow rate of O₂ (i.e. flow rate regulation device) arranged downstream from the means for measurement of the flow rate of oxygen;
it comprises means for regulation of the NO content which are configured to allow a user to select a desired NO content and supply the said desired NO content to the control means;
the control means are configured to calculate a flow rate of NO/N₂ mixture to be supplied corresponding to the desired NO content, and to control the NO/N₂ flow rate control means in order to supply the said flow rate of NO/N₂ mixture calculated;
the control means are configured to calculate the flow rate of NO/N₂ mixture to be supplied from the desired NO content, the flow rate of oxygen measured by the means for measurement of the flow rate of oxygen, and an NO content in the NO/N₂ mixture conveyed by the main NO line;
it also comprises an information display screen, preferably a digital screen with touch control;
it comprises a digital display screen with touch control, i.e. a touch panel screen;
the means for regulating the NO content comprise at least one touch selection key displayed on the display screen, preferably touch selection keys which are displayed on the display screen;
the said at least one touch selection key is configured to allow the user to set or select a desired NO content of between 1 and 80 ppmv;
the NO content in the NO/N₂ mixture conveyed by the main NO line is between 100 and 1,000 ppmv;
in the absence of actuation of the means for actuation by the user, the control means are also configured to ensure normal operation of the device by controlling the NO/N₂ flow rate control means such as to convey the NO/N₂ mixture via the main gas circuit, between the said at least one NO input port and the main output orifice;
the main NO line is connected fluidically to two NO input ports which are arranged in parallel;
when they are supplied with electrical energy (i.e. electric current), the control means are configured to receive the activation signal which is emitted after actuation of the means for actuation by the user;
the backup NO line and the main O₂ line are connected fluidically to one another at a junction site situated downstream for the means for regulating the flow rate of O₂ of the main O₂ line, and downstream from the second solenoid valve of the backup NO line;
the O₂ flow rate control means comprise a flow rate sensor or a differential pressure sensor;
the main O₂ line is configured to cooperate pneumatically with the pneumatic valve by means of a section of duct which connects the main O₂ line downstream from the second control valve fluidically to the said pneumatic valve;
the first control valve is of the all-or-nothing type;
the first control valve is configured to control the circulation of the flow of oxygen in the main O₂ line;
the first control valve is controlled by means for actuation which can be actuated by the user, such as a member of the medical staff, when he wishes to start or stop the backup circuit;
the means for actuation comprise a rotary switch or the like which can be actuated by the user;
the means for regulation of the NO content comprise at least one selection key with touch actuation which is displayed on the display screen, i.e. one or more touch selection keys, configured to allow a user to set or select a desired NO content, and preferably touch selection keys which are displayed on the display screen. In other words, the selection or setting of the desired concentration of NO is thus carried out by the user pressing his finger on the selection key(s) with touch actuation displayed on the display screen;
the control means are configured to control the display(s) on the display screen;
the control means comprise at least one (micro)processor, for example a microcontroller;
the control means comprise at least one electronic board comprising the said at least one microprocessor;
the control means comprise at least one (micro)processor which implements at least one algorithm, for example for data processing, calculation or the like;
in response to the receipt of the activation signal, the control means are also configured to act on the second solenoid valve, in order to interrupt any circulation of NO/N₂ mixture in the backup NO line;
the first solenoid valve is arranged on the main NO line downstream from the NO/N₂ flow rate control means, and on the backup NO line of the backup NO circuit downstream from the pneumatic valve;
the first solenoid valve with a plurality of ways cooperates with the backup NO line via a connection duct which connects the backup NO line to one of the ways of the first solenoid valve;
the first solenoid valve is arranged on the portion of duct downstream from the main NO line, between the NO/N₂ flow rate control means and the main output orifice;
the first solenoid valve is controlled by the control means in order to permit or stop any passage of NO/N₂ mixture obtained from the NO/N₂ flow rate control means;
the junction site is situated downstream from the first solenoid valve;
the backup NO line comprises a device with a calibrated orifice arranged downstream from the pneumatic valve;
a first pressure regulator is arranged on the backup NO line upstream from the pneumatic valve;
a second pressure regulator is arranged on the main O₂ line, between the second pneumatic control valve and the means for measurement of the flow rate of oxygen;
an NO flow rate indicator device is arranged on the backup NO line, upstream from the junction site of the main O₂ line and the backup NO line;
an O₂ flow rate indicator device is arranged on the main O₂ line, downstream from the device or means for regulation of the O₂ flow rate;
the O₂ flow rate indicator device comprises a ball rotameter;
the means for regulation of the O₂ flow rate comprise a rotary disc with calibrated orifices or the like;
the means for regulation of the O₂ flow rate are configured to adjust the flow rate of the flow of O₂ to between 5 and 20 L/min;
it comprises means for selection of the flow rate which cooperate with the means for regulation of the flow rate of O₂ in order to select a desired flow rate of O₂;
the means for selection of the flow rate can be actuated by the user;
the means for selection of the flow rate comprise a rotary knob or the like;
according to another embodiment, the means for selection of the flow rate comprises a key or the like displayed on a display screen in order to select the flow rate of O₂;
it comprises means for data storage, for example a computer memory or the like;
it comprises a rigid housing in which some or all of the elements of the NO supply device are arranged;
the main O₂ line is configured to cooperate pneumatically with the pneumatic valve in order to permit the passage of the NO/N₂ mixture in the backup NO line, after the user has actuated the means for actuation, i.e. in response to actuation by the user of the means for actuation;
in the absence of actuation of the means for actuation by the user, the control means are also configured to ensure normal operation of the device by controlling the NO/N₂ flow rate control means such as to convey the NO/N₂ mixture via the main gas circuit, between the said at least one NO input port and the main output orifice;
the touch selection keys allow the user to make a selection from amongst a plurality of proposed NO contents, i.e. to set or select a desired NO content, for example from amongst a plurality of predefined NO contents;
alternatively, the touch selection keys comprise “+’ and “−” keys which make it possible to increase or decrease a given NO value per step, for example by 1 ppmv, or another increment (e.g. by 2, by 3, . . . or by 5 ppmv);
the said desired NO content which is chosen or selected by the user by action on the said at least one touch selection key is supplied to the control means, preferably by means of digital action;
the display screen is a colour or black and white display;
the electrical energy supply means, i.e. for supply of electric current, comprise means for electrical connection to the mains (110/220V), for example an electric cable provided with an electric plug or the like, and/or an electric battery which is preferably rechargeable;
the display screen is supported by the housing;
the secondary output, i.e. a backup output, such as an output port or orifice, is configured to be connected fluidically to a manual insufflator, such as a manual breathing bag or BAVU, in particular via a flexible fluidic connection duct, i.e. a flexible tube or the like;
the main NO line comprises at least one upstream portion of duct, and preferably two upstream portions of duct arranged in parallel;
the backup NO circuit is supplied with NO/N₂ by the said at least one upstream portion of duct of the main NO line, and preferably the two upstream portions of duct arranged in parallel;
the backup NO circuit is connected fluidically to the said at least one upstream portion of duct, and preferably the two upstream portions of duct arranged in parallel, via at least one section of NO input piping, and preferably two sections of NO input piping in parallel connected fluidically to the two upstream portions of duct;
the backup NO circuit is connected fluidically to the main NO line, upstream from the NO/N₂ flow rate control means, in particular to the said upstream portions of duct of the main NO line, such as to be supplied with NO/N₂;
the or the two sections of NO input piping are supplied with NO/N₂ by the or the two NO input ports.

According to another aspect, the invention also concerns an installation for administering therapeutic gas containing NO to a patient (P) comprising an NO supply device according to the invention, in particular as described above, supplied with NO/N₂ mixture by at least one pressurised gas container, and with oxygen by a pressurised oxygen container, the said NO supply device supplying:
either an NO/N₂ mixture via the main output orifice 210, to a respiratory gas circuit 3 connected to a medical ventilator 2;
or an NO/N₂/O₂ mixture via the secondary orifice 141, to a manual insufflator or BAVU.

According to the embodiment concerned, the therapeutic gas administration installation according to the invention can comprise one or more of the following characteristics:
the said at least one pressurised gas container contains the NO/N₂ mixture;
the said at least one pressurised gas container contains an NO/N₂ mixture containing from 100 to 1000 ppmv of NO, the remainder being nitrogen;
the pressurised oxygen container contains medical oxygen;
the container(s) is/are a gas cylinder/gas cylinders;
the gas cylinder(s) contain(s) an NO/N₂ mixture or medical oxygen at a pressure of at least 150 bar, or at least 180 bar;
the respiratory gas circuit comprises an inspiratory branch and an expiratory branch;
the inspiratory branch and the expiratory branch are connected fluidically to one another via a junction part, such as a part in the form of a “Y”;
the junction part is connected to fluidically to a respiratory interface, such as an endotracheal tube or a respiratory mask;
the inspiratory branch and the expiratory branch comprise flexible tubes;
a gas humidifier is arranged on the respiratory gas circuit, in particular on the inspiratory branch;
the inspiratory branch comprises a flow rate sensor which is connected electrically to the NO supply device, in particular to the control means;
the flow rate sensor is arranged on the inspiratory branch upstream from the NO injection site;
the flow rate sensor is of the mass type or with pressure differential;
a gas collection line connects the NO supply device fluidically to the respiratory gas circuit, preferably in the vicinity of the junction part, i.e. the part in the form of a “Y”;
the medical ventilator and the NO supply device are supplied electrically by electrical energy supply means, i.e. at least one source of electric current, in particular means for electrical connection to the mains (110/220V), for example an electrical cable provided with an electric plug or the like, and/or an electric battery, which is preferably rechargeable;
the secondary output, i.e. the backup output, of the NO supply device is connected fluidically to a manual insufflation bag, preferably via a connection duct, such as a flexible tube.

According to another aspect, the invention also concerns a method for treatment of a person, known as the patient, suffering from a pathology or a medical condition giving rise to acute pulmonary arterial hypertension, in particular pulmonary vasoconstrictions in adults, adolescents or infants, including the newborn and babies, for example in order to treat persistent pulmonary hypertension of the newborn (PPHN) in a newborn, baby, infant or the like, or pulmonary hypertension in a person undergoing cardiac surgery, wherein administration by inhalation is provided to the said patient needing it, of a gaseous mixture containing oxygen (preferably >21% by volume), nitrogen and NO (preferably <100 ppmv), the said gaseous mixture being supplied by a therapeutic gas administration installation according to the invention comprising a device or apparatus for supplying NO according to the invention, in particular as described above and/or hereinafter, which NO supply device is supplied with NO/N₂ mixture by at least one pressurised gas container, and preferably a plurality of pressurised gas containers 5, such as NO/N₂ cylinders, and with oxygen by at least one pressurised oxygen container, such as an O₂ cylinder, and which NO supply device being able to supply: either an NO/N₂ mixture, via the main output orifice, to the respiratory gas circuit connected to a medical ventilator of the therapeutic gas administration installation according to the invention, or an NO/N₂/O₂ mixture via the secondary orifice, to a manual insufflator, in particular a manual insufflation bag or BAVU.

### BRIEF DESCRIPTION OF THE DRAWINGS

The invention will now be better understood thanks to the following detailed description, provided by way of non-limiting illustration, with reference to the appended figures in which:

FIG. 1 represents an embodiment of an installation for administering therapeutic gas to a patient P incorporating an NO supply device;

FIG. 2 schematises an embodiment of the backup circuit of an NO supply device according to the prior art;

FIG. 3 schematises an embodiment of the internal architecture of an NO supply device according to the present invention; and

FIG. 4 is a detailed diagram of the means for controlling the flow rate of NO/N₂ of the device of FIG. 3.

### DESCRIPTION OF THE PREFERRED EMBODIMENTS

FIG. 1 schematises an embodiment of an installation 100 for administering therapeutic gas, i.e. a gaseous mixture based on NO, to a patient P, incorporating an NO supply device 1, such as the one according to the present invention.

More specifically, in this case it comprises two pressurised gas containers 5, arranged in parallel, each containing a gaseous mixture of NO and nitrogen (N₂), i.e. a NO/N₂ mixture, typically containing from 250 to 1000 ppmv of NO and nitrogen (N₂), which is also subjected to a pressure which can be as much as 180 bars or more, for example a NO/N₂ mixture, containing 450 ppmv or 800 ppmv of NO. Gas containers 5 of this type are commonly known as NO cylinders 5.

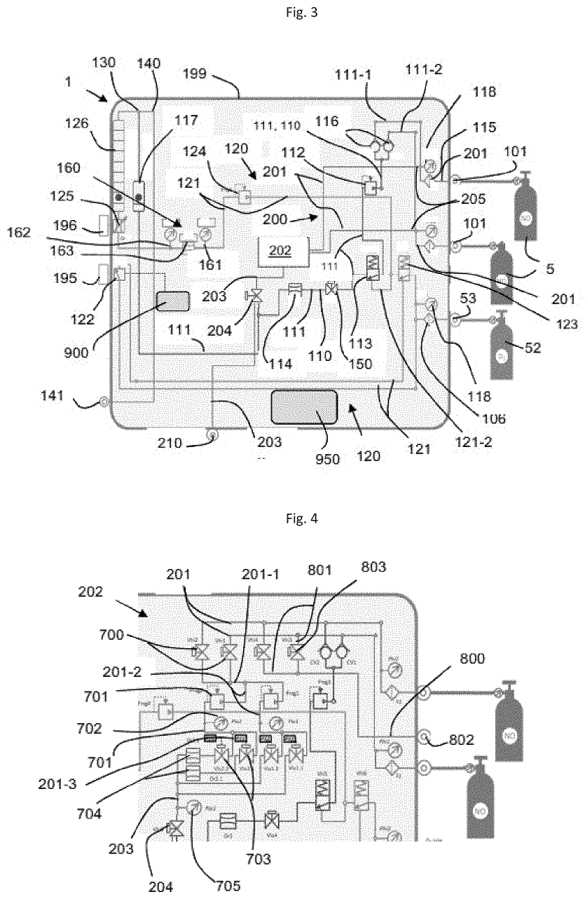

The NO cylinders 5 supply the NO/N₂ mixture to an NO supply device 1, such as the one according to the invention, the internal architecture of which is illustrated in FIG. 3 and FIG. 4. They are connected fluidically to the gas supply device 1 by NO supply lines 50, i.e. gas piping, such as flexible tubes or the like. Each NO supply line 50 is connected to an NO input port 101 of the NO supply device 1, in order to supply a main gas circuit 200 inside the housing 199 of the NO supply device (cf. FIG. 3).

The NO supply device 1 also comprises an oxygen input port 53 which is connected fluidically, via an oxygen supply line 51, such as a flexible tube of the like, to a source of oxygen, for example a pressurised oxygen container 52 typically an O₂ cylinder, or, alternatively, the hospital network, i.e. oxygen supply piping provided in the hospital building where the patient P is being cared for.

The NO cylinders 5 and the O₂ cylinder 52 are equipped with a gas distribution valve 55, preferably incorporating gas expansion means, i.e. an RDI or valve with an integrated expansion device, such as to be able to control the flow rate and/or the pressure of the gas which they supply. The gas distribution valve 55 is preferably protected against impacts by a protective cowl.

In addition, the installation 100 also comprises a medical ventilator 2, i.e. a respiratory assistance apparatus, which supplies a flow of respiratory gas containing at least 21% by volume of oxygen, such as air or an oxygen/nitrogen mixture (NO/N₂) to the patient P.

The medical ventilator 2 is fluidically connected to the patient P via a respiratory gas circuit 3 which in this case has two respiratory branches 30, 31, since it comprises an inspiratory branch 30, i.e. a gas supply line which is used to convey the respiratory gas to the patient P, and an expiratory branch 31 which is used to recuperate the gas enriched with CO₂ exhaled by the patient P.

The two respiratory branches 30, 31 are typically flexible tubes made of polymer or the like. The two respiratory branches 30, 31 are firstly connected to the medical ventilator 2, and secondly connected to one another at a junction part 32, typically a part in the form of a “Y”, which is in fluid communication with a respiratory interface 4 which supplies gas to the patient P, such as an endotracheal tube or the like.

It will be appreciated that the medical ventilator 2 and the NO supply device 1 are normally supplied electrically by one or more sources of electric current, in particular their components which require electrical energy in order to operate, in particular the means 900 for controlling the NO supply device 1 and the system for controlling the medical ventilator 2, i.e. an electronic board with a microprocessor/microprocessors, or any other component, in particular the motorised internal turbine which supplies the flow of air or the like, i.e. the respiratory gas. The source of electric current can be the mains (110/220V) and/or an electric battery, which is preferably rechargeable.

As can be seen, the NO supply device 1 makes it possible to inject the NO/N₂ mixture into the inspiratory branch 30, via an NO injection duct 11 which opens in the inspiratory branch 30 into an injection site 8, such as to provide there a mixture of the flow of NO/N₂ and of the flow of respiratory gas containing at least 21% O2, i.e. air or an oxygen/nitrogen mixture, supplied by the medical ventilator 2.

The NO supply device 1 comprises a main output orifice 210 situated at the outlet of its main gas circuit 200 via which the flow of NO/N₂ exits from the housing 199 of the NO supply device 1, and penetrates into the NO injection duct 11. The NO injection duct 11 is connected fluidically to the main output orifice 210, for example via a connector or the like.

The therapeutic gaseous mixture obtained thus contains oxygen (>21% by volume), nitrogen, and a concentration of NO which is variable and adjustable, typically of between 1 and 80 ppmv, as a result of the dilution which takes place during the mixture of the gaseous flows. It will be appreciated that inevitable impurities can be found in the gas, but they are not desirable, in particular when the flow of gas obtained from the ventilator 2 is atmospheric air rather than a mixture of O₂/N₂.

Advantageously, a gas humidifier 6 is also provided, which in this case is arranged on the inspiratory branch 30 downstream from the injection site 8 used to humidify the flow of therapeutic gas, i.e. an NO/N₂/O₂ mixture, by the addition of water vapour, before it is inhaled by the patient P, which makes it possible to avoid or limit the drying of the respiratory tracts of the patient P during his treatment by inhalation of the gas. According to another embodiment, the gas humidifier 6 could also be arranged upstream from the injection site 8. Depending on the case, the expiratory branch 31, used to receive the exhaled gases rich in CO₂, can comprise one or more optional components, such as, for example, a CO₂ elimination device, i.e. a CO₂ trap, such as a hot container or the like, which makes it possible to eliminate the CO₂ present in the gases exhaled by the patient, a filter or the like.

As shown in FIG. 1, on the inspiratory branch 30, upstream from the injection site 8, a flow rate sensor 7 is also provided, for example of the mass type or with pressure differential, connected to the NO supply device 1, in particular to the control means 900 of the said NO supply device 1, via a line 71 for measurement of the flow rate of respiratory gas used to measure the flow rate of gas obtained from the ventilator 2 within the inspiratory branch 30. Determining this flow rate of the ventilator (Qv) makes it possible in particular to regulate the passage of the NO through the NO supply device 1, in particular to be able to select the flow rate of NO/N₂ mixture to be injected according to the desired NO content, the composition of the NO/N₂ mixture obtained from the cylinders, and the flow rate of gas (i.e. air or air/O₂) obtained from the ventilator 2.

In addition, a gas collection line 33 can also be provided, connecting the NO supply device 1 fluidically to the respiratory gas circuit 3, preferably in the vicinity of the part in the form of a “Y” 32, for example at approximately 10 to 20 cm upstream from the part in the form of a “Y” 32, used to collect gas samples and verify the conformity thereof with the gaseous mixture required, to be administered to the patient P.

More specifically, the NO supply device 1 comprises an internal main gas circuit 200 to convey the NO/N₂ mixture entering via the gas input port(s) 101 as far as the NO injection duct 11. This main gas circuit 200 comprises means 202 for controlling the flow rate of NO/N₂ (cf. FIG. 3 and FIG. 4), such as valves, calibrated orifices etc., controlled by the control means 900 of the NO supply device 1, typically one or more microprocessors arranged on an electronic board, the operation of which is explained hereinafter. All these components are arranged in a housing 199, i.e. In a rigid external carcass.

In addition, the NO supply device comprises a so-called backup circuit 110, designed to supply an adjustable flow rate of O₂ and a fixed flow rate of NO, in order to be able to ensure a supply of NO, even in the case of a fault or the like, as described hereinafter.

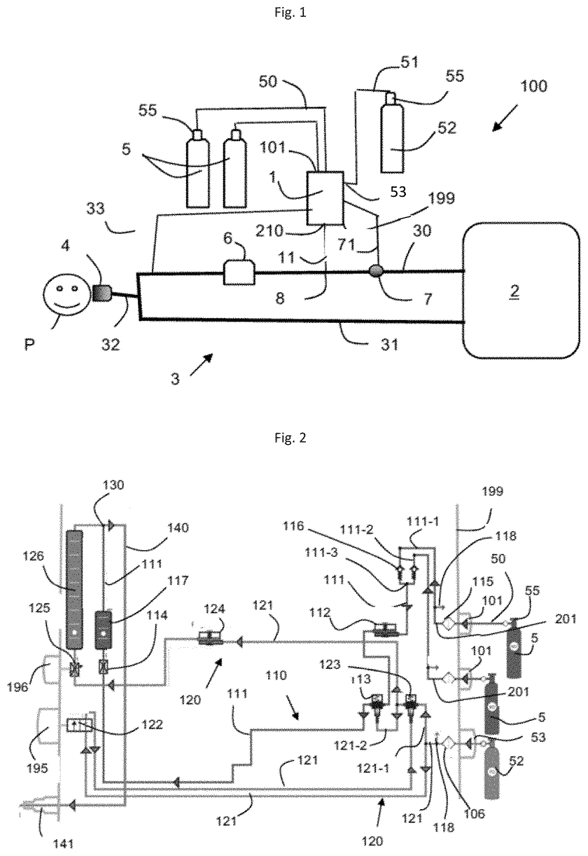

Thus, FIG. 2 schematises an embodiment of the backup circuit 110 or secondary circuit of a conventional NO supply device 1. Above all, it must be specified that, in FIG. 2, the main gas circuit 200 of the NO supply device 1, which conveys the NO/N₂ mixture during the normal operation of the device 1, i.e. when there is no fault or the like, is not represented in order not to encumber the diagram unnecessarily, and complicate understanding thereof. However, this main gas circuit 200, which is arranged in the housing 199, like the one illustrated in FIG. 3 or FIG. 4, comprises a main NO line 201, 203 comprising at least one upstream portion of duct 201, i.e. in this case two upstream portions of duct 201 arranged in parallel, since 2 cylinders of NO 5 are connected to the NO supply device 1, as illustrated in FIG. 2 to FIG. 4, and also at least one downstream portion 203.

As can be seen, the backup circuit 110, 120, the operation of which can be entirely pneumatic, which is also arranged in the housing 199, comprises a backup NO circuit 110 and a backup O₂ circuit 120, each comprising gas piping, passages or ducts, making it possible to convey the different gases or gaseous mixtures into the housing 199.

The backup NO circuit 110 is supplied with NO/N₂ by at least one section of NO input piping 111-1, 111-2, i.e. in this case two sections of NO input piping 111-1, 111-2 arranged in parallel, which themselves are supplied by the two NO input ports 101 to which the NO cylinders 5 are connected, via the supply line 50, as explained hereinafter.

The section(s) of input piping 111-1, 111-2 are connected fluidically to the upstream portions of duct 201 (partly represented in FIG. 2) downstream from a filter 115 situated immediately downstream from each NO input port 101 in the upstream portion(s) of piping 201, as shown in detail in FIG. 3.

A non-return valve 116 is arranged in each section of input piping 111-1, 111-2. These two sections of input piping 111-1, 111-2 join downstream from the non-return valves 116, in order to form a common backup NO line, starting from a junction site 111-3.

Each upstream portion of duct 201 also comprises a pressure measurement means 118, such as a pressure sensor with a strain gauge (silicon pressure sensor) or the like, used to verify the existence of a pressure in the upstream portion of duct 201 reflecting the presence of a cylinder connected, and therefore also to verify the quantity of gas which it contains so as to decide whether the cylinder must be changed (if it is almost empty), and switch to the other cylinder of gas. The said pressure measurement means 118 are situated downstream from the filter 115.

It will be appreciated that, according to another embodiment, the device 1 needs to comprise only a single NO input port 101 and thus a single upstream portion of duct 201 and a single section of input piping 111-1. In this case, only a single oxygen supply line 51, such as a flexible tube or the like, can be connected fluidically thereto, which is supplied with NO/N₂ mixture by one or more NO cylinders 5.

The backup NO line 111 makes it possible to convey the pressurised NO/N₂ mixture, obtained, via the input piping sections 111-1, 111-2, from the upstream portions of duct 201 supplied by the NO cylinders 5 which supply the NO/N₂ mixture at a pressure of approximately 3 to 6 bars relative at the output of the RDI 55.

It can additionally be seen that the backup NO line 111 also comprises a first pressure regulator 112, downstream from the junction site 111-3, in order to reduce or control the pressure of the NO/N₂ mixture, for example in order to supply a reduced pressure equal to approximately 3.2 bars relative, and a pneumatic valve 113 which makes it possible to control the circulation of the NO/N₂ mixture. This pneumatic valve 113 is situated downstream from the first pressure regulator 112, taking into consideration the direction of circulation of the gas, in the knowledge that the gaseous NO/N₂ mixture circulates in the direction going from each gas input port 101 towards the pressure regulator 112.

The opening of the pneumatic valve 113, and thus the passage of the flow of NO/N₂, is controlled by the pressure of the flow of oxygen supplied by the main O₂ line 121, via the second section of duct 121-2, as explained hereinafter.

Downstream from the pneumatic valve 113, the backup NO line 111 comprises a device with a calibrated orifice 114 or the like, making it possible to regulate the flow of gas, i.e. of the NO/N₂ mixture, and a device for indicating the flow rate of NO 117 making it possible to check that the flow rate of NO/N₂ mixture is equal to the fixed value anticipated, for example approximately 230 mL/min.

In addition, the backup oxygen circuit for its part comprises a main O₂ line 121 which is used to convey the oxygen into the housing 199, which is supplied by the O₂ input port 102 to which the O₂ cylinder 52 is connected, via the supply line 51 which supplies the gaseous oxygen at a pressure typically of between 3 and 6 bar.

The main O₂ line 121 is connected at a junction site 130 to the backup NO line 111, downstream from the NO flow rate indicator device 117, forming there a common line 140, such as to provide a mixture of the flow of oxygen with an adjustable flow rate, as explained hereinafter, and of the NO/N₂ flow which has a fixed flow rate of approximately 230 mL/min for example.

As can be seen in FIG. 2, the main O₂ line 121 also comprises a filter 106, which is arranged immediately downstream from the O₂ input port 102, as well as a first control valve 122 and a second, pneumatic control valve 123. It also comprises means 118 for measurement of pressure, such as a pressure sensor with a strain gauge, as explained above.

The first control valve 122, which is typically of the all-or-nothing type, makes it possible to control the circulation of the flow of oxygen in the main O₂ line 121. It is controlled by means 195 for actuation, such as a rotary switch, a push-button, a selection key or the like, which can be actuated by the user, such as a member of the medical staff, when he wishes to start or stop the backup circuit 110, 120.

A first section of duct 121-1 is connected fluidically to the main O₂ line 121, between the O₂ input port 102, in particular downstream from the filter 106, and the first control valve 122. The first section of duct 121-1 makes it possible to control the second, pneumatic control valve 123 pneumatically.

For as long as the first control valve 122 is closed, the gaseous pressure which is exerted in the portion of the main O₂ line 121 situated upstream from the first control valve 122, and thus also in the first section of duct 121-1, will act on the second, pneumatic control valve 123, maintaining it closed, which will prevent any passage of oxygen through this second control valve 123.

When the user actuates the means 195 for actuation, the first control valve 122 opens, and thus permits the passage of the gas in the direction of the second control valve 123. As a result of the pressure of the oxygen reaching the second control valve 123, a sufficient force will be applied to make it possible to open the control valve 123, as a result of the pressure provided by the first section of duct 121-1, which will then open the second control valve 123, and thus allow the oxygen to pass into the downstream part of the oxygen circuit 120 which is situated downstream from the second control valve 123.

The oxygen then continues its route in the main O₂ line 121, passing through a second pressure regulator 124 in order to reduce or control the pressure of the flow of oxygen, for example in order to obtain a pressure of 1.6 bar absolute relative. The flow rate of the flow of O₂ can then be adjusted, for example to between 5 and 20 L/min, by means for regulation of the flow rate of O₂₁₂₅, such as a rotary disc with calibrated orifices or the like, arranged on the main O₂ line 121 downstream from the second pressure regulator 124.

The selection of the desired flow rate of O₂ can be carried out by the user via a flow rate selection means 196, such as a rotary knob or the like, supported by the housing 199 of the device (or, according to another embodiment, selection of a flow rate of O₂ via a key or the like displayed on a display screen 950), which flow rate selection means 196 cooperates with the means for regulation of the flow rate of O₂ 125.

The flow rate of oxygen can then be checked via an O₂ flow rate indicator device 126, such as a ball rotameter or the like, arranged downstream from the device or means for regulation of the O₂ flow rate 125. The flow of oxygen obtained can then be mixed with the flow of NO/N₂ starting from the junction site 130, where the main O₂ line 121 is connected to the backup NO line 111, as already mentioned.

Since this backup circuit 110, 120 is entirely pneumatic, in order to control the circulation of NO/N₂ within the backup NO line 111, a second section of duct 121-2 is provided, which is connected fluidically to the main O₂ line 121 between the second control valve 123 and the second pressure regulator 124. This second section of duct 121-2 controls the pneumatic valve 113 of the backup NO circuit 110, thanks to the oxygen pressure which it contains.

More specifically, for as long as the second control valve 123 is closed, the pressure of the flow of oxygen is not exerted in the second section of duct 121-2, and therefore the pneumatic valve 113 of the NO circuit 110 also remains closed.

On the contrary, when the second control valve 123 opens after actuation by the user of the means for actuation 195 and opening of the first control valve 122, as explained above, the flow of pressurised oxygen will be able to pass through the second control valve 123, then spread downstream therefrom, in particular into the second section of duct 121-2, in order then to act pneumatically on the pneumatic valve 113 of the NO circuit 110, and open it, thus opening up the passage for the NO/N₂ mixture and its circulation in the downstream part of the backup NO line 111 situated downstream from the pneumatic valve 113.

The first control valve 122, the second control valve 123 and the pneumatic valve 113 are for example controlled pneumatic valves of the shutter and spring type.

In other words, the activation of the means for activation 195 by the user leads to quasi-synchronised and/or quasi-simultaneous release of the flows of NO/N₂ and O₂ mixture within the backup NO circuit 110 and the backup oxygen circuit 120, thus resulting in a mixture thereof starting from, and downstream from, the junction site 130, i.e. in the common backup line 140 which opens at a backup output 141, i.e. a secondary output, which for example is supported by a connection connector or the like, which can be connected to a manual gas insufflator, i.e. a manual breathing bag or the like.

It is understood that the operation of this backup or secondary gas circuit 110,120 is entirely pneumatic, since it uses only gas ducts and pneumatic valves in order to control the flows of gas.

However, as already explained, an entirely pneumatic circuit of this type is not ideal, since the concentration of NO of the gaseous mixture obtained (i.e. NO/N₂₊₀₂) from the junction site 130, and thus in the common backup line 140 situated downstream, varies according to the flow rate of oxygen regulated by the user, since the flow rate of NO is fixed, whereas that of the oxygen is adjustable.

Thus, for a NO/N₂ gaseous mixture with 800 ppmv of NO (remainder nitrogen) the concentration of NO varies between 8 and 32 ppmv for a flow rate of oxygen of between 5 and 20 L/min, whereas, for a NO/N₂ mixture with 450 ppmv of NO, the concentration of NO varies between 4.5 and 18 ppmv for the same flow rate of oxygen.

However, having a concentration of NO which varies according to the flow rate of O₂ selected is not acceptable, since it is necessary to be able to provide the patient with a given dose, i.e. a fixed quantity of NO corresponding to a concentration which is efficient for treating the patient's pathology, in particular within the context of ventilation of the patient by a manual breathing bag or BAVU in the place and instead of normal ventilation by the medical ventilator 2.

It is therefore necessary to be able to make the concentration of NO of the gaseous NO/N₂/O₂ mixture obtained independent from the flow rate of oxygen, when the backup mode is activated by the user, and ventilation of the patient by means of a manual breathing bag must be put into effect.

For this purpose, in the improved NO supply device 1 as illustrated in FIG. 3 and FIG. 4, which schematise an embodiment of the internal architecture, an NO supply device 1 according to the present invention is provided.

Firstly, the NO supply device 1 of FIG. 3 comprises entirely pneumatic elements of the backup gas circuit 110, 120 of FIG. 2, which bear the same references and are therefore not explained again hereinafter.

Next, as can be seen in FIG. 3, the NO supply device 1 of the invention also comprises the main internal gas circuit 200 which, during normal operation of the device 1, is used to supply the NO/N₂ mixture via the NO injection duct 11, to the inspiratory branch 30 of the ventilation circuit 3, which is connected to, and supplied by, the medical ventilator 2 with respiratory gas, i.e. air or an N₂/O₂ mixture, as explained above in association with FIG. 1.

The main gas circuit 200 comprises at least one upstream portion of duct 201 i.e. in this case two upstream portions of duct 201 to which the sections of input piping 111-1, 111-2 are connected fluidically, at a connection site 205 which is situated between the filter 115 and the non-return valve 116 of each section of input piping 111-1, 111-2, as represented in FIG. 3. It will be appreciated that, if only one section of input piping 111-1 is provided, according to the embodiment concerned, as explained above, then only one upstream portion of duct 201 is necessary.

The portion(s) of upstream duct 201 make(s) it possible to convey the NO/N₂ mixture obtained from the sections of input piping 111-1, 111-2 as far as NO/N₂ flow rate control means 202 which make it possible to control the flow rate of the flow of NO/N₂ mixture which must then be supplied to the injection duct 11. An embodiment of the NO/N₂ flow rate control means 202 is described in FIG. 4.

After its passage into the NO/N₂ flow rate control means, the gaseous flow is conveyed by a downstream portion of duct 203 of the main NO line 201, 203, which brings it and supplies it to the injection duct 11, via the main output orifice 210 which is at the output end of the downstream portion of duct 203 of the main gas circuit 200.

A first solenoid valve 204 with a plurality of ways, in this case 3 ways, is arranged on the downstream portion of duct 203 of the main NO line 201, 203, between the means for controlling the flow rate of NO/N₂ 202 and the main output orifice 210, i.e. it is connected by 2 of these 3 ways to the downstream portion of duct 203, such as to control the circulation of the gas in the downstream portion of duct 203. It is also connected, via its third way, to the backup NO line 111, downstream from the pneumatic valve 113, which itself is situated downstream from the first pressure regulator 112, taking into consideration the direction of circulation of the gas, i.e. the NO/N₂ mixture, in the backup NO line 111. In this case, the first solenoid valve 204 cooperates with the backup NO line 111 via a connection duct which connects the backup NO line 111 to the third way of the first solenoid valve 204.

In addition, as shown in FIG. 3, the backup NO line 111 comprises a second solenoid valve 150 with 2 ways, which is normally in the open position, i.e. it allows the gas to circulate within the backup NO line 111, and the device with a calibrated orifice 114 or the like which makes it possible to regulate the gaseous flow rate, i.e. the flow rate of the NO/N₂ mixture, which in FIG. 2 is arranged in the vicinity of the NO flow rate indicator device 117. The second solenoid valve 150 is thus situated between the pneumatic valve 113 and the first solenoid valve 204 with 3 ways.

The second solenoid valve 150 is normally in the open position, but it can be controlled by the control means 900, in particular in the case of activation of the backup mode in the event of a slight fault, as explained hereinafter, or if it is necessary to ventilate a patient by means of the manual insufflator in order to carry out manoeuvres of alveolar induction for example.

According to another embodiment (not schematised), the first solenoid valve 204 with 3 ways and the second solenoid valve 150 with 2 ways can be replaced by a single solenoid valve with 5 ways, which would be installed instead of, and in the place of, the first solenoid valve 204 with 3 ways (the second solenoid valve 150 would then be eliminated).

Finally, in addition, the NO supply device 1 according to the invention comprises means 160 for measurement of the flow rate of oxygen arranged on the main O₂ line 121 between the second pressure regulator 124, and the device or means for regulation of the flow rate of O₂ 125.

These means for measurement of the flow rate of oxygen 160 can comprise a flow rate sensor or a differential pressure sensor, the pressure ports 161, 162 of which are connected upstream and downstream from a flow restrictor 163, such as a Venturi system, a calibrated orifice or the like, in order to measure a pressure differential (ΔP), i.e. a loss of load, making it possible to deduce therefrom a flow rate of oxygen (Q_{O2}).

FIG. 4 represents a detailed embodiment of the means for controlling the flow rate of NO/N₂ which are used to control the flow rate of NO/N₂ within the main gas circuit 200 arranged in the housing 199 of the NO supply device 1.

As can be seen, the two upstream portions of duct 201 of the main gas circuit 200 are joined in order to form a common section 201-1 conveying the NO/N₂ mixture. Each upstream portion of duct 201 comprises a first control valve 700, which is arranged upstream from the site of junction of the two upstream portions of duct 201, which is used to permit or stop the circulation of the flow of NO/N₂ mixture supplied by one or the other of the upstream portions of duct 201.

The comment section 201-1 is then divided into two secondary sections 201-2 arranged in parallel with one another, which each comprise a pressure regulator device 701 and a pressure sensor 702.

The two secondary sections 201-2 are divided in turn into a plurality of sub-sections 201-3, which are also arranged in parallel with one another, i.e. in this case two sub-sections 201-3. Each sub-section 201-3 comprises a second control valve 703. The sub-sections 201-3 also comprise additional means for controlling the flow rate 704, for example one or more calibrated orifices or the like, making it possible to adjust the flow rate to a desired flow rate value.

It will be appreciated that the means for controlling the flow rate of NO/N₂ 202 can comprise more sub-sections 201-3, which in particular are each equipped with a control valve 703 and additional flow rate control means 704, typically a calibrated orifice, in order to make it possible to provide a greater variety of flow rates of NO if necessary. Preferably, the control valve(s) 703 is/are a valve/valves of the all-or-nothing type (AON). The flow rate of gas circulating therein is fixed, and depends on the pressure of the pressure regulator 701, i.e. an expansion device, and on the cross-section of the calibrated orifices.

In fact, the means 900 for controlling the NO supply device 1, such as an electronic board bearing one or more microprocessor(s) which implement one or more algorithms, control the first control valves 700 and the second control valves 703, in order to direct the gases into appropriate sections making it possible to obtain the desired dose of NO.

The desired NO content is selected by the user via means for selection of the NO content (not schematised) arranged on the device, for example one or more keys, cursors, selection knobs, in particular a rotary knob or the like, which make it possible to select or set a desired NO content.

Advantageously, the NO supply device 1 according to the invention is equipped with an information display screen 950, typically a digital screen, preferably a touch screen, in colour or black and white, which is configured to display the value of the desired NO content and/or other information, such as the NO content in the NO cylinders 5, or also the flow rate values of NO or O₂, and for example also the concentration of NO₂, the flow rate supplied by the ventilator, etc.

According to one embodiment, the information display screen 950 is a digital screen with touch control, and the means for selection of the NO content are keys with selection by touch actuation, displayed on the touch digital display screen 950. The selection or setting of the concentration of NO desired is thus carried out by the user pressing his finger on the keys with selection by touch actuation displayed on the display screen 950.

Once the NO content has been selected, the means 900 for controlling the NO supply device 1 calculate the flow rate of NO/N₂ mixture to be supplied, in particular depending on other parameters, such as the NO content of the NO/N₂ mixture obtained from the cylinders 5, and also the flow rate of respiratory gas (typically air or air/O₂) supplied by the ventilator 2, and then determine which control valves 700, 703 must be opened or closed in order to direct the gases into the sections suitable for obtaining the dose of NO set.

The sub-sections 201-3 then join, upstream from the first solenoid valve 204 with 3 ways, in a single line which forms the downstream portion of duct 203 of the main gas circuit 200. This can comprise a pressure sensor 705 or the like, upstream from the first solenoid valve 204, in order optionally to be able to correct the flow rate, which depends on the variation of pressure (ΔP) of the gas when it passes through the additional flow rate control means 704, typically one or more calibrated orifices.

In addition, it can also be seen that the device 1 for supplying NO according to the invention also comprises a purging line 800 which communicates with the exterior via a purging orifice 802 arranged in the housing 199. The purging line 800 branches into two purging sections 801 which are connected to the two upstream portions of duct 201, in order to be able to carry out gaseous purging of these two upstream portions of duct 201. Each purging section 801 comprises a purging valve 803 which is controlled by the control means, such as to be able to eliminate all the types of NO₂ which can form in the residual gas present in the piping by means of oxidation of the NO molecules by the oxygen, when the device 1 is not operating, i.e. when it is not being used.

In normal operation, i.e. when the user has not actuated the means for actuation 195, the NO/N₂ mixture is conveyed in the main gas circuit 200 of the NO supply device 1 according to the invention, via the means for controlling the flow rate of NO/N₂ 202, from one of the gas input ports 101 as far as the main output port 210 which supplies the NO/N₂ mixture to the NO injection duct 11, such as to be able then to inject at the desired concentration of NO, into the inspiratory branch 30 of the ventilation circuit 3 connected to the medical centre later 2, as explained above in association with FIG. 1 and FIG. 4.

In this case, the second solenoid valve 150 with 2 ways is in the “normally open” position, i.e. it does not interrupt the passage of gas in the backup NO line 111, and the first solenoid valve 204 with 3 ways is in the “normal operation” position which permits the passage of the gaseous flow into the downstream portion of duct 203 as far as the main output orifice 210.

The first solenoid valve 204 is controlled by the control means 900 in order to permit or stop any passage of NO/N₂ mixture obtained from the means for control (i.e. regulation) of the flow rate of NO 202.

In the event of a serious or severe fault of the NO supply device 1 according to the invention resulting in a loss of electrical energy and/or stoppage of operation of the control means 900 of the device 1, or of the medical ventilator 2, in particular involving software or another programme or control algorithm implemented by one or more microprocessors of the said control means, the second solenoid valve 150 with 2 ways remains in the “normally open” position, and the first solenoid valve 204 with 3 ways remains in the “normal operation” position, such as to permit circulation of the gaseous flow in the downstream portion of duct 203, as far as the main output orifice 210 After actuation by the user of the means for actuation 195, this makes possible transition to backup mode of an entirely pneumatic type, as explained in association with FIG. 2 This therefore results in supply by the backup NO line 111, downstream from the NO flow rate indicator device 117, of a flow of NO/N₂ with a fixed flow rate of approximately 230 mL/min for example which is mixed (at 130) with the flow of oxygen with an adjustable flow rate coming from the main O₂ line 121.

The backup gaseous mixture formed by NO/N₂/O₂ thus obtained can then be conveyed by the common line 140 as far as the secondary or backup output 141, which then supplies this backup gaseous mixture to a manual breathing bag, also known as a manual insufflator or BAVU. Then, the ventilation of the patient is thus carried out via the manual breathing bag, in order to assure a supply of NO to the patient despite the severe fault or the like.

On the other hand, if the fault of the NO supply device 1 according to the invention is a slight fault which does not result in a total loss of electrical energy and/or a stoppage of operation of the control means 900 of the device 1, and/or if the medical staff decides to carry out ventilation of the patient via a manual breathing bag, while desiring to comply with the dose of NO, then the NO supply device 1 according to the invention, and more particularly the control means 900 and the display screen 950, continue to be supplied with electrical energy by the electrical energy supply means, and therefore the control means 900 continue to be able to operate.

In this case, the user wishing to put the device 1 into backup mode or the mode of ventilation by BAVU, in order to assure a supply of gas to the patient via a manual breathing bag or BAVU, as previously will actuate the means for actuation 195, which will then give rise to opening of the first control valve 122, thus permitting circulation of the flow of oxygen in the main O₂ line 121 as far as the second control valve 123, which will thus also open, and allow the oxygen to pass into the downstream part of the oxygen circuit 120, situated downstream from the second control valve 123, as explained above in association with FIG. 2.

The means 160 for measurement of the flow of oxygen which are arranged on the main O₂ line 121 downstream from the second pressure regulator 124 will then be able to measure the flow rate of oxygen (Q_{O2}) and transmit this measurement of flow rate of O₂ (i.e. signal or value) to the control means 900, in order to make it possible to calculate the flow rate of NO/N₂ to be supplied, as explained hereinafter.

The flow of oxygen can then continue its route as far as the junction site 130, where the O₂/NO/N₂ mixture takes place, via the means for regulation of the flow rate of O₂ 125 and the O₂ flow rate indicator device 126, such as a ball rotameter or the like, as already explained.

However, in this case, the means for actuation 195 are connected electrically to the control means 900, and are configured to supply thereto actuation information, i.e. an activation signal corresponding to the position of the means for actuation 195, that is in backup mode (i.e. backup activated) or in normal mode (i.e. backup not activated). This activation signal is supplied electrically to the control means 900 of the NO supply device 1 according to the invention, which receives and processes it in order to determine whether the means for actuation 195 have been actuated by the user in order to trigger the backup mode or mode of ventilation by BAVU, for the purpose of carrying out ventilation of the patient via a manual breathing bag connected to the secondary output, i.e. the backup output 141, of the NO supply device 1 according to the invention, for example via a flexible tube.

If this is the case, the means 900 for controlling the NO supply device 1 according to the invention which continue to be supplied with electrical energy (i.e. electric current), react, in response to this signal, to the means for controlling the flow rate of NO/N₂ 202, in order to supply the NO/N₂ mixture at the desired flow rate, and to the first solenoid valve 204 with 3 ways, in order to permit the passage of the flow of NO/N₂ from the downstream portion of duct 203 of the main gas circuit 200 to the downstream part of the backup NO line 111 which is situated downstream from the first solenoid valve 204, and conveys the flow of NO/N₂ via the NO flow rate indicator device 117, as far as the mixture site 130, where the main O₂ line 121 and the backup NO line 111 join, as already explained.

Thus, with the NO/N₂ mixture being produced within the NO/N₂ flow rate control means 202, it is possible to set the most suitable flow rate of NO. This flow rate of NO/N₂ mixture to be supplied by the NO/N₂ flow rate control means 202 can be calculated by the control means 900 of the NO supply device 1 according to the invention from the final content of NO desired, the composition of the NO/N₂ mixture in the cylinders 5, and the flow rate of oxygen measured by the means for measurement of the flow rate of oxygen 160 which are arranged on the main O₂ line 121 downstream from the second pressure regulator 124.

In this case, the final content of NO desired is thus selected by the user via the means for regulation of NO content arranged on the device, i.e. preferably one or more digital keys with actuation by means of the fingers, displayed on the digital display screen 950, which also continues to be supplied with electric current.

Being able to use a NO/N₂ mixture produced within the means for controlling the flow rate of NO/N₂ 202, in the case of activation of the backup circuit and in the absence of a complete fault of the device 1, i.e. in the case of a slight fault, and/or if it is wished to ventilate the patient via a manual breathing bag, has the advantage of guaranteeing greater precision of concentration of NO of the backup mixture supplied to the patient, since the said final content of NO can be regulated by the user via the means for regulation of the content of NO (i.e. choice or selection), and the control means 900 use this value of regulated NO content, the concentration of NO in the NO/N₂ mixture supplied by the NO cylinders 5, and the value of O₂ flow rate measured, in order to determine the flow rate of NO/N₂ to be supplied, and act accordingly on the means for controlling the flow rate of NO/N₂ 202, in particular the first control valves 700 and the second control valves 703, so as to supply the appropriate flow rate of NO/N₂ by controlling the passage of the gas, in particular into the secondary sections 201-2, the pressure regulator device 701, the two secondary sections 201-2, and the sub-sections 201-3 comprising the additional means for controlling the flow rate 704, for example calibrated orifices or the like, thus making it possible to adjust the flow rate to a desired flow rate value.

In other words, thanks to the incorporation of the means for measurement of the flow rate of oxygen 160 on the main O₂ line 121, downstream from the second pressure regulator 124, which measure the flow rate of oxygen (Q_{O2}) and transmit this O₂ flow rate measurement to the control means, the control means can calculate an NO/N₂ flow rate set point to be supplied by the NO/N₂ flow rate control means 202, also taking into account not only the NO set point which is regulated by the user, but also the concentration of the NO in the NO/N₂ mixture obtained from the NO cylinders 5.

This results in being able to obtain from the mixture site 130 a final mixture with a precise content of NO, since the flow rate of NO/N₂ mixture is no longer fixed (e.g. equal to 230 mL/min), but is adjustable according to the other parameters which have an influence.

However, in this case, it is essential to prevent the flow of oxygen which passes through the second control valve 123 and acts on the pneumatic valve 113 from giving rise to an excessive supply of NO via the backup NO line 111, through the second solenoid valve 150 with 2 ways. For this purpose, when they receive the activation signal obtained from the means for actuation 195, which corresponds to transition to backup mode, the control means 900 of the NO supply device 1 according to the invention are also configured to act additionally on the second solenoid valve 150 with 2 ways, which is normally in the open position, in order to close it, and thus prevent the NO/N₂ mixture from being able to circulate within the backup NO line 111, and supply the downstream part of the backup NO line 111 which is situated downstream from the first solenoid valve 204.

The NO/N₂/O₂ mixture thus obtained is then, as previously, conveyed by the common line 140 which opens at a backup output 141, i.e. a secondary output, in order to supply the backup gaseous mixture formed by NO/N₂/O₂, and then to be able to supply it, via a flexible duct or the like, to a manual breathing bag to carry out manual ventilation of the patient with the gaseous NO/N₂/O₂ mixture with the desired dose.

On the other hand, in the case of a serious fault of the device 1 with an electrical supply defect, no activation signal is generated by the means for actuation 195, and/or is not used/processed by the control means 900, and/or the display screen can not operate, therefore in the case of activation of the backup mode via the means for actuation 195, the device 1 goes back into entirely pneumatic backup mode, as explained in association with FIG. 2.

The NO supply device 1 according to the invention has a higher level of safety, by making it possible to adjust the NO content more precisely in the case of a slight failure or fault, without loss of electrical supply, or when the user wants to change to manual ventilation of the patient by BAVU.

In general, the NO supply device 1 according to the invention is particularly suitable for use within an installation 100 for administration of therapeutic gas based on NO (<100 ppmv), O₂ (>21% by volume) and nitrogen to a patient P, such as the installation 100 in FIG. 1, implemented in order to treat one or more patients suffering from a pathology or medical condition giving rise to acute pulmonary arterial hypertension, in particular pulmonary vasoconstrictions in adults or children, including the newborn, for example in order to treat PPHN in a newborn, or pulmonary hypertension in a person undergoing cardiac surgery.

In other words, according to another aspect, the invention thus also concerns a method for treatment of a person, known as the patient, suffering from a pathology or medical condition giving rise to rise to acute pulmonary arterial hypertension, in particular pulmonary vasoconstrictions in adults or children, including the newborn, for example in order to treat persistent pulmonary hypertension of the newborn (PPHN), in a newborn, baby or the like, or pulmonary hypertension in a person undergoing cardiac surgery, wherein the said patient needing it is administered by inhalation with a gaseous mixture containing oxygen (preferably >21% by volume), nitrogen and NO (preferably <100 ppmv), the said gaseous mixture being supplied by an installation 100 for administration of therapeutic gas according to the invention comprising an NO supply device 1 according to the invention, which NO supply device 1 is supplied with an NO/N₂ mixture by at least one pressurised gas container 5, preferably a plurality of pressurised gas containers 5, such as NO/N₂ cylinders, and with oxygen by at least one pressurised oxygen container 52, such as an O₂ cylinder, and which NO supply device 1 is able and designed to supply: either an NO/N₂ mixture via the main output orifice 210, to the respiratory gas circuit 3 which is connected to a medical ventilator 2 of the installation 100 for administration of therapeutic gas, or an NO/N₂/O₂ mixture via the secondary orifice 141, to a manual insufflator, in particular a manual breathing bag or BAVU.

In general, within the context of the present invention all the terms “means for” are considered to be totally equivalent and can be replaced by the term “device for” for example the terms “control means” can be replaced by “control device”, and the terms “measurement means” can be replaced by “measurement device”, etc.

1. NO supply device comprising:
a main gas circuit comprising at least one main NO line connecting at least one NO input port fluidically to a main output orifice, in order to convey a NO/N₂ mixture of the said at least one NO input port to the said main output port; and
a backup circuit comprising a backup NO circuit and a backup O₂ circuit, wherein:
the backup NO circuit comprises a backup NO line which is supplied with NO/N₂ mixture obtained from the said at least one NO input port; and
the backup O₂ circuit comprises a main O₂ line which is in fluid communication with an O₂ input port, the main O₂ line being connected fluidically to the backup NO line in order to form a common line which is in fluid communication with a secondary output, supplying an NO/N₂/O₂ mixture,
wherein the main NO line comprises means for controlling the flow rate of NO/N₂ which are controlled by control means, and the main NO line is connected fluidically to the backup NO line, downstream from the means for controlling the flow rate of NO/N₂, by means of a first solenoid valve with a plurality of ways.

2. The device according to claim 1, wherein the first solenoid valve has at least 3 ways.

3. The device according to claim 1, wherein the first solenoid valve with a plurality of ways is controlled by the control means such as to direct the flow of NO/N₂ mixture obtained from the NO/N₂ flow rate control means:
either into a downstream portion of the backup NO line which is in fluid communication with the common line in fluid communication with the secondary output;
or into a downstream portion of the main NO line which is in fluid communication with the main output orifice.

4. The device according to claim 1, wherein the backup NO line comprises a pneumatic valve which makes it possible to control the circulation of the NO/N₂ mixture in the backup NO line, and a second solenoid valve which is controlled by the control means.

5. The device according to claim 1, wherein the main O₂ line comprises:
a first control valve controlled by means for actuation which can be actuated by a user in order to control the circulation of the flow of oxygen into the said main O₂ line, the said means for actuation also being configured to supply an activation signal to the control means in response to actuation of the said means for actuation by the user; and
a second pneumatic control valve which is arranged downstream from the first control valve.

6. The device according to claim 5, wherein the first solenoid valve has at least 3 ways, and wherein the control means are configured to control the NO/N₂ flow rate control means such as to regulate the flow rate of the NO/N₂ mixture in the main gas circuit, and to control the first solenoid valve in response to the receipt of the activation signal supplied after actuation of the means for actuation by the user.

7. The device according to claim 5, wherein the main O₂ line additionally comprises:
means for measurement of the flow rate of oxygen which are arranged downstream from the first control valve, and are configured to provide at least one measurement of flow rate of oxygen to the control means; and
means for regulation of the flow rate of O₂ arranged downstream from the means for measurement of the flow rate of oxygen.

8. The device according to claim 1, wherein:
the device comprises means for regulation of the NO content which are configured to allow a user to select a desired NO content and supply the said desired NO content to the control means; and
the control means are configured to calculate a flow rate of NO/N₂ mixture to be supplied corresponding to the desired NO content, and to control the NO/N₂ flow rate control means in order to supply the said flow rate of NO/N₂ mixture calculated.

9. The device according to claim 8, wherein the main O₂ line additionally comprises:
means for measurement of the flow rate of oxygen which are arranged downstream from the first control valve, and are configured to provide at least one measurement of flow rate of oxygen to the control means; and
means for regulation of the flow rate of O₂ arranged downstream from the means for measurement of the flow rate of oxygen, and
wherein the control means are configured to calculate the flow rate of NO/N₂ mixture to be supplied from the desired NO content, the flow rate of oxygen measured by the means for measurement of the flow rate of oxygen, and an NO content in the NO/N₂ mixture conveyed by the main NO line.

10. The device according to claim 1, wherein the secondary output is configured to be connected fluidically to a manual insufflator.

11. The device according to claim 8, further comprising a digital display screen with touch control, and the means for regulating the NO content comprise at least one touch selection key displayed on the display.

12. The device according to claim 1, wherein the backup NO circuit is connected fluidically to the main NO line upstream from the NO/N₂ flow rate control means.

13. The device according to claim 5, wherein the means for actuation comprise a rotary switch which can be actuated by the user.

14. The device according to claim 1, wherein the control means comprise at least one microprocessor.

15. Installation for administering therapeutic gas containing NO to a patient comprising an NO supply device according to claim 1, supplied with NO/N₂ mixture by at least one pressurized gas container and with oxygen by a pressurized oxygen container, the said NO supply device supplying:
either an NO/N2 mixture via the main output orifice, to a respiratory gas circuit connected to a medical ventilator;
or an NO/N2/O₂ mixture via the secondary orifice, to a manual insufflator or BAVU.

16. Installation for administering therapeutic gas containing NO to a patient comprising an NO supply device according to claim 2, supplied with NO/N₂ mixture by at least one pressurized gas container and with oxygen by a pressurized oxygen container, the said NO supply device supplying:
either an NO/N2 mixture via the main output orifice, to a respiratory gas circuit connected to a medical ventilator;
or an NO/N2/O₂ mixture via the secondary orifice, to a manual insufflator or BAVU.

17. Installation for administering therapeutic gas containing NO to a patient comprising an NO supply device according to claim 3, supplied with NO/N₂ mixture by at least one pressurized gas container and with oxygen by a pressurized oxygen container, the said NO supply device supplying:
either an NO/N2 mixture via the main output orifice, to a respiratory gas circuit connected to a medical ventilator;
or an NO/N2/O₂ mixture via the secondary orifice, to a manual insufflator or BAVU.

18. Installation for administering therapeutic gas containing NO to a patient comprising an NO supply device according to claim 4, supplied with NO/N₂ mixture by at least one pressurized gas container and with oxygen by a pressurized oxygen container, the said NO supply device supplying:
either an NO/N2 mixture via the main output orifice, to a respiratory gas circuit connected to a medical ventilator;
or an NO/N2/O₂ mixture via the secondary orifice, to a manual insufflator or BAVU.

19. Installation for administering therapeutic gas containing NO to a patient comprising an NO supply device according to claim 5, supplied with NO/N₂ mixture by at least one pressurized gas container and with oxygen by a pressurized oxygen container, the said NO supply device supplying:
either an NO/N2 mixture via the main output orifice, to a respiratory gas circuit connected to a medical ventilator;
or an NO/N2/O₂ mixture via the secondary orifice, to a manual insufflator or BAVU.

20. Installation for administering therapeutic gas containing NO to a patient comprising an NO supply device according to claim 6, supplied with NO/N₂ mixture by at least one pressurized gas container and with oxygen by a pressurized oxygen container, the said NO supply device supplying:
either an NO/N2 mixture via the main output orifice, to a respiratory gas circuit connected to a medical ventilator;
or an NO/N2/O₂ mixture via the secondary orifice, to a manual insufflator or BAVU.
